(19) United States
(12) Patent Application Publication
(10) Pub. No.: US 2015/0202392 A1
(43) Pub. Date: Jul. 23, 2015

(54) METERING ELEMENT FOR AN INHALATION DEVICE AND INHALATION DEVICE COMPRISING A METERING ELEMENT

(71) Applicant: SANOFI SA, Vemier (CH)

(72) Inventor: Stefan Mayer, Freiburg im Breisgau (DE)

(21) Appl. No.: 14/414,741

(22) PCT Filed: Jul. 16, 2013
(86) PCT No.: PCT/EP2013/065011

§ 371 Date: Jan. 14, 2015

(30) Foreign Application Priority Data
Jul. 20, 2012 (EP) 12177298.2

(51) Int. Cl. A61M 15/00

(52) U.S. Cl. CPC A61M 15/0068; A61M 15/0021; A61M 15/0091; A61M 2202/064

(57) ABSTRACT

A metering element for an inhalation device is provided, comprising at least one opening being configured to receive a substance, wherein the opening comprises a width and an entire height, wherein the height extends along a longitudinal axis of the metering element and the width extends across the longitudinal axis, and wherein the continuous width of the opening is less than the entire height of the opening. Furthermore, an assembly for an inhalation device is provided, comprising a metering element and a sealing member wherein a removal of substance from the metering chamber upon mechanical cooperation of the metering element with the sealing member is prevented.

### CROSS REFERENCE TO RELATED APPLICATIONS

The present application is a U.S. National Phase Application pursuant to 35 U.S.C. §371 of International Application No. PCT/EP2013/065011 filed Jul. 16, 2013, which claims priority to European Patent Application No. 12177298.2 filed Jul. 20, 2012. The entire disclosure contents of these applications are herewith incorporated by reference into the present application.

### TECHNICAL FIELD

The present disclosure relates to a metering element for an inhalation device and an inhalation device comprising a metering element.

### BACKGROUND

A metering element for an inhalation device is known from document WO 2009/065707 A1. This application relates to a metering device which can be activated by the suction airflow of a user for inhaling a powdery substance which is arranged in a supply chamber.

### SUMMARY

It is an object of the present invention to provide a metering element for an inhalation device having improved properties.

According to one aspect of the disclosure, a metering element for an inhalation device is provided. The metering element comprises at least one opening being configured to receive a substance, wherein the opening comprises a width and an entire height, wherein the height extends along a longitudinal axis of the metering element and the width extends across the longitudinal axis. Preferably, the continuous width of the opening is less than the entire height of the opening.

According to a further aspect of the disclosure, a metering element for an inhalation device is provided being configured to be moved in a moving direction. The metering element comprises at least one opening being configured to receive a substance, wherein the opening comprises a width and an entire height, wherein the height extends parallel to the moving direction of the metering element and the width extends across the moving direction of the metering element. Preferably, the continuous width of the opening is less than the entire height of the opening.

The metering element may be configured as a rod. The metering element may have a rectangular cross-section. Alternatively, the metering element may have a circular cross-section.

The metering element may be configured to move axially in a moving direction. The moving direction may be a direction along the longitudinal axis of the metering element. In an alternative embodiment, the moving direction may be a direction tilted with respect to the longitudinal axis, in particular perpendicular to the longitudinal axis of the metering element. Furthermore, the metering element may rotate with respect to its longitudinal axis. For delivering a dose of substance, the metering element may be moved along the longitudinal axis towards a dispensing end of the device. After use, the metering element may be moved away from the dispensing end of the device.

The continuous width of the opening may be a width which is free of interruptions. Such an interruption may be, for example, a grating. For example, the continuous width may extend from a boundary of the opening to an interruption. Alternatively, the continuous width may be a width which extends between two interruptions. Alternatively, the continuous width may be a width which extends between two opposing boundaries of the opening. The entire width of the opening may extend between two opposing boundaries of the opening. Particularly, the entire width may extend between a left and a right boundary of the opening. Particularly, when the opening is free of any interruptions, the continuous width coincides with the entire width of the opening.

The entire height may be the distance between the most proximal point of the opening and the most distal point of the opening. The most proximal point of the opening may be a point which is furthest away from a dispensing end of the device. The most distal point of the opening may be a point which is nearest to the dispensing end of the device. A continuous height may be a height which is free of any interruptions. For example, the continuous height may be a height between the most proximal point of the opening and an interruption. Alternatively, the continuous height may be a height between the most distal point of the opening and an interruption. When the opening is free of any interruptions, the continuous height may coincide with the entire height of the opening.

Preferably, due to the shape of the opening a high accuracy of dosing may be achieved. Due to the shape of the opening the substance may adhere to the metering element. In particular, due to the shape of the opening no substance may drop out unintentionally through the opening. Thereby, the delivery of a wrong dose of substance may be inhibited. Thereby, the safety for a user may be increased.

Preferably, the metering element is configured to measure a sub-quantity of a substance from a total quantity of substance. In particular, the metering element may receive a sub-quantity of substance via the opening. The substance may be a powder. The substance may be a medicament.

In one embodiment, the metering element comprises one opening. The opening may be shaped such that the continuous width of the opening is less than its height. Particularly, the opening may have a narrow configuration. For example, the opening may have the form of an ellipse.

In an alternative embodiment, the metering element comprises a plurality of openings. The openings may have the form of an ellipse. In one embodiment, the metering element may comprise at least two openings. For example, the metering element comprises four openings being arranged in terms of a square. Alternatively, the openings may be arranged in terms of a rhomb. In an alternative embodiment, the metering element may comprise a different number of openings, such as three, five or six.

According to one embodiment, the opening may comprise at least two segments. For example, the opening comprises two, four or six segments.

In one embodiment, the metering element comprises a grating. The grating may be configured to reduce the continuous width of the opening. Particularly, the grating may interrupt the entire width of the opening. Furthermore, the grating may interrupt the entire height of the opening. The opening may have the form of an ellipse. Alternatively, the opening may have the form of a circle. The grating may be arranged above the opening. In particular, the grating may be align with an outer surface of the metering element.

Preferably, the grating may define two or more segments of the opening. For example, the grating may define four segments of the opening. Alternatively, the grating may define six segments of the opening. Each of these segments has a width and a height. The width of one segment may coincide with the continuous width of the opening. The height of one segment may coincide with the continuous height of the opening. In one embodiment, the grating may have the form of a star. In an alternative embodiment, the grating may have the form of a cross.

In a preferred embodiment, the metering element comprises a metering chamber. Preferably, the metering chamber is configured to receive a sub-quantity of a total quantity of substance via the opening.

The metering chamber may be a cavity in the metering element. The cavity may be conical. The metering chamber may extend through the metering element without any interruption.

Preferably, the opening is arranged eccentrically on the metering element with respect to the longitudinal axis. On rotation and axial movement of the metering element the opening, respectively the metering chamber, moves through an accumulation of substance helicoidally. Thereby, the metering chamber acts as a shovel gathering substance. Thereby, an adequate filling of the metering chamber with substance may be achieved on rotation and axial movement of the metering element.

Preferably, a dropping-out of an amount of the sub-quantity of substance from the metering chamber is inhibited. Particularly, the opening is shaped such that a dropping-out of substance from the metering chamber is inhibited. In particular, a dropping-out of substance from the metering chamber may be inhibited due to the opening having a continuous width smaller than its entire height. For example, the interior surface of the metering chamber with respect to its volume may be large, compared to, for example, a metering chamber with an opening having a continuous width being equal to its entire height. Thereby, the adherence of the substance to the interior surface of the metering chamber may be sufficient for the substance to not drop out of the metering chamber unintentionally.

According to a further aspect of the disclosure, an assembly for an inhalation device is provided. The assembly comprises a metering element and a sealing member. The sealing member may comprise a sealing lip. The metering element may comprise an opening being configured to receive a substance, wherein the opening may comprise a width and a height, wherein the continuous width of the opening is less than the entire height of the opening. Preferably, the orientation of the metering element and the sealing member may be such that the height of the opening extends perpendicular to the orientation of the sealing lip.

In particular, the height of the opening may extend perpendicular to the orientation of the sealing lip irrespective of the orientation of the longitudinal axis of the metering element. The width of the opening may extend along, respectively perpendicular, to the orientation of the sealing lip.

According to a further aspect of the disclosure, an assembly for an inhalation device is provided. Preferably, the inhalation device comprises a metering element and a sealing member. The metering element may be configured as previously described. Preferably, a removal of substance from the metering chamber upon mechanical cooperation of the metering element with the sealing member is prevented.

The inhalation device may be configured for delivering a plurality of sub-quantities of substance to a patient.

In one embodiment, the sealing member may comprise a main body and a sealing lip. The sealing member may mechanically cooperate with the metering element. In particular, the sealing lip may mechanically cooperate with the metering element.

In one embodiment, the sealing lip may be aligned perpendicular to the moving direction of the metering element.

During a movement of the metering element in the moving direction, respectively along the longitudinal axis of the device a removal of substance from the metering chamber upon mechanical cooperation of the metering element with the sealing member may be prevented. Particularly, the sealing lip does not immerge into the metering chamber.

Preferably, the opening may be configured such that the sealing member does not immerge into the metering chamber. In particular, the sealing lip may not immerge into the metering chamber due to the opening having a continuous width smaller than its entire height.

Thereby, the accuracy of dosage may be improved. In particular, it may be inhibited that the sealing lip on immerging into the metering chamber unintentionally removes an amount of substance from the metering chamber.

Preferably, the sealing member is configured for sealing the metering chamber. Preferably, the sealing member comprises an opening. The metering element may be guided through the sealing member, in particular through the opening of the sealing member, when the metering element is moved along its moving direction.

Preferably, the metering element is configured to axially move along a longitudinal axis of the inhalation device. Furthermore, the metering element is configured to rotate about the longitudinal axis of the device.

Preferably, the assembly for the inhalation device comprises a storage chamber.

Preferably, the storage chamber is configured to contain a quantity of substance. In particular, the storage chamber may be configured to contain a plurality of sub-quantities of substance.

Preferably, the metering element is configured to transport a sub-quantity of substance out of the storage chamber. In particular, the metering element is configured to transport a sub-quantity of substance out of the storage chamber via the metering chamber.

Preferably, the metering element is configured to rotate and axially move with respect to the storage chamber. The metering element may axially move from a first position inside the storage chamber to a second position outside the storage chamber. The first position may be a proximal position of the metering element furthest away from the dispensing end of the device. In the first position the metering element is at least partially positioned in the storage chamber. In particular, at least the metering chamber is fully dipped in the storage chamber. The second position may be a distal position of the metering element nearest to the dispensing end of the device. In the second position, the metering element is positioned outside of the storage chamber. In particular, the metering chamber is positioned outside of the storage chamber.

Preferably, the metering element gathers a sub-quantity of substance from the storage chamber on rotation. The metering element may gather a sub-quantity of substance when it is in the storage chamber. Preferably, the metering element transports a sub-quantity of substance out of the storage chamber on axial movement.

In a preferred embodiment, the assembly for an inhalation device comprises a powder channel. Preferably, the metering element is configured to transport a sub-quantity of substance from the storage chamber to the powder channel. During an inhalation, the sub-quantity of substance may flow through the powder channel. Preferably, the flow profile of the substance in the powder channel is influenced by the shape of the opening. Furthermore, the flow profile of the substance in the powder channel may be influenced by the shape of the metering chamber. Preferably, the substance comprises a fine particle fraction. Preferably, the flow profile has an influence on the composition of the fine particle fraction of the substance.

According to a further aspect of the disclosure, an inhalation device comprising the above disclosed assembly is provided.

The term “substance”, as used herein may mean a pharmaceutical formulation containing at least one pharmaceutically active compound, for example for the treatment of obstructive airway or lung diseases such as asthma or chronic obstructive pulmonary disease (COPD), local respiratory tract oedema, inflammation, viral, bacterial, mycotic or other infection, allergies, diabetes mellitus.

The active pharmaceutical compound is preferably selected from the group consisting of active pharmaceutical compounds suitable for inhalation, preferably antiallergenic, antihistamine, anti-inflammatory, antitussive agents, bronchodilators, anticholinergic drugs, and combinations thereof.

The active pharmaceutical compound may for example be chosen from:

an insulin such as human insulin, e.g. a recombinant human insulin, or a human insulin analogue or derivative, a glucagon-like peptide (GLP-1) or an analogue or derivative thereof, or exendin-3 or exendin-4 or an analogue or derivative of exendin-3 or exendin-4;

an adrenergic agent such as a short acting β2-agonists (e.g. Salbutamol, Albuterol, Levosalbutamol, Fenoterol, Terbutaline, Pirbuterol, Procaterol, Bitolterol, Rimiterol, Carbuterol, Tulobuterol, Reproterol), a long acting β2-agonist (LABA, e.g. Arformoterol, Bambuterol, Clenbuterol, Formoterol, Salmeterol), an ultra LABA (e.g. Indacaterol) or another adrenergic agent (e.g. Epinephrine, Hexoprenaline, Isoprenaline (Isoproterenol), Orciprenaline (Metaproterenol));

a glucocorticoid (e.g. Beclometasone, Budesonide, Ciclesonide, Fluticasone, Mometasone, Flunisolide, Betamethasone, Triamcinolone);

an anticholinergic agent or muscarinic antagonist (e.g. Ipratropium bromide, Oxitropium bromide, Tiotropium bromide);
a mast cell stabilizer (e.g. Cromoglicate, Nedocromil);

a xanthine derivative (e.g. Doxofylline, Enprofylline, Theobromine, Theophylline, Aminophylline, Choline theophyllinate);

an eicosanoid inhibitor, such as a leukotriene antagonist (e.g. Montelukast, Pranlukast, Zafirlukast), a lipoxygenase inhibitor (e.g. Zileuton) or a thromboxane receptor antagonist (e.g. Ramatroban, Seratrodast);

a phosphodiesterase type-4 inhibitor (e.g. Roflumilast);

an antihistamine (e.g. Loratadine, Desloratadine, Cetirizen, Levocetirizine, Fexofenadine);

an allergen immunotherapy (e.g. Omalizumab);

a mucolytic (e.g. Carbocisteine, Erdosteine, Mecysteine);

an antibiotic or antimycotic;

or a combination of any two, three or more of the above-mentioned compound classes or compounds (e.g. Budesonide/Formoterol, Fluticasone/Salmeterol, Ipratropium bromide/Salbutamol, Mometasone/Formoterol);

or a pharmaceutically acceptable salt or solvate or esters of any of the above named compounds.

Pharmaceutically acceptable salts are for example acid addition salts and basic salts. Acid addition salts are e.g. a chloride, bromide, iodide, nitrate, carbonate, sulfate, methylsulfate, phosphate, acetate, benzoate, benzenesulfonate, fumarate, malonate, tartrate, succinate, citrate, lactate, gluconate, glutamate, edetate, mesylate, pamoate, pantothenate or a hydroxy-naphthoate salt. Basic salts are for example salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology. Pharmaceutically acceptable ester may for example be acetates, propionates, phosphates, succinates or etabonates.

Pharmaceutically acceptable solvates are for example hydrates.

Further features and refinements become apparent from the following description of the exemplary embodiments in connection with the figures.

### BRIEF DESCRIPTION OF THE FIGURES

FIG. 1 schematically shows a sectional view of an inhalation device.

FIG. 2 schematically shows a cross-section of a metering element.

FIGS. 3A and 3B schematically show a cooperation of a sealing member with a metering element.

FIGS. 4A to 4F schematically show different embodiments of a metering element.

Like elements, elements of the same kind and identically acting elements may be provided with the same reference numerals in the figures.

### DETAILED DESCRIPTION

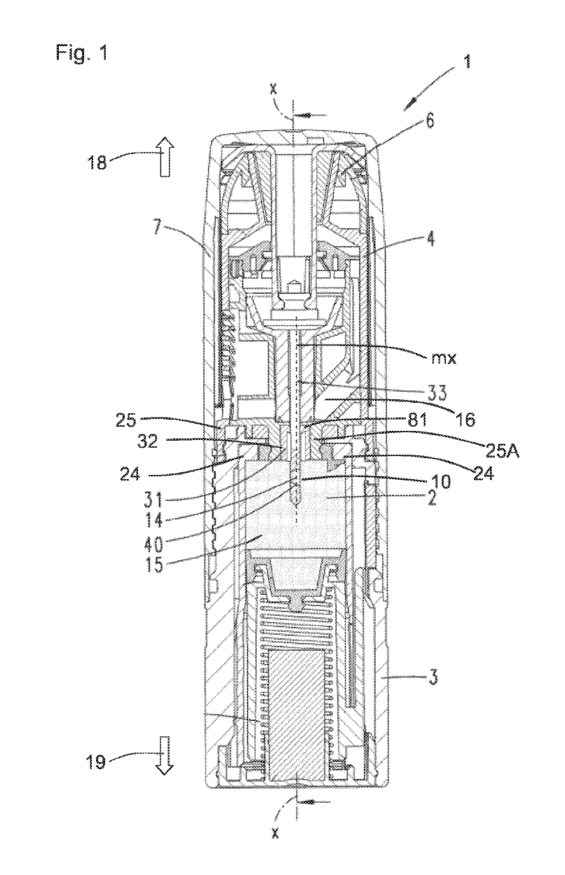

FIG. 1 shows a sectional view of an inhalation device 1. The inhalation device 1 is configured to be activated by a suction airflow generated by a user. The inhalation device 1 comprises a housing 3. Furthermore, the device 1 comprises an outer cylinder 4. The outer cylinder 4 is secured against axial movement with respect to the housing 3. The outer cylinder 4 is rotatable with respect to the housing 3.

Furthermore, the inhalation device 1 comprises a mouthpiece 6. Via the mouthpiece 6, air is sucked into the inhalation device 1. The inhalation device 1 further comprises a cap 7. The cap 7 may be configured as a screw cap. The cap 7 is used for covering the mouthpiece 6. The cap 7 may be rotatable about a main longitudinal axis x of the inhalation device 1 in a first direction with respect to the housing for screwing the cap onto the device 1 and in a second direction with respect to the housing 3 for unscrewing the cap from the device 1. The outer cylinder 4 is rotationally connected to the cap 7. In particular, the outer cylinder 4 follows a rotation of the cap 7 with respect to the housing 3. For a detailed description of the components of the inhalation device 1 and their mechanical cooperation it is referred to document WO 2009/065707 A1, the entire content of which is explicitly incorporated by reference into the present description, in particular as far as the operation of the device 1 is concerned.

The device 1 further comprises a storage chamber 15. The storage chamber 15 holds at least one dose of a substance 2. In particular, the storage chamber 15 may hold a plurality of doses of a substance 2. The substance 2 may comprise a drug. The substance 2 may comprise a powder.

The storage chamber 15 is terminated by a chamber sealing 24. In particular, the side of the storage chamber which is faced towards the mouthpiece is terminated by the chamber sealing 24. The device 1 further comprises a rotary part 25. The rotary part 25 is connected in a rotationally fixed manner to the outer cylinder 4. Accordingly, the rotary part 25 follows a rotation of the outer cylinder and, hence, of the cap 7 about the main longitudinal axis x with respect to the storage chamber 15.

The chamber ceiling 24 comprises a central through opening. A cylindrical portion 25A of the rotary part 25 passes through the central through opening of the chamber ceiling 24.

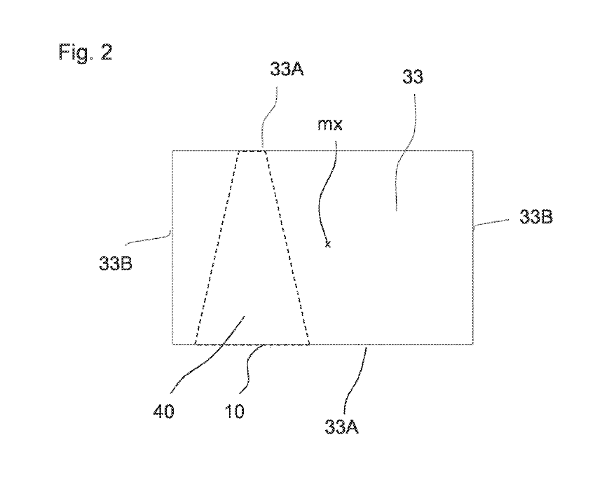

The inhalation device 1 further comprises a metering element 33. The metering element 33 may comprise a metering rod. The metering element 33 may have a circular or a non-circular cross-section. For example, the metering element 33 may have a rectangular cross-section. FIG. 2 shows a cross-sectional view of the metering element 33 in a plane perpendicular to the longitudinal axis x. As can be seen in FIG. 2, the metering element 33 may comprise a first side 33A and a second side 33B. The first side 33A may be broader than the second side 33B. The dimension of the first side 33A may comprise a length from 3 mm to 10 mm. For example, the length of the first side 33A may be 6 mm. The dimension of the second side 33B may comprise a length from 0.5 mm to 3 mm. For example, the dimension of the second side 33B may comprise a length of 1.5 mm.

The metering element 33 comprises a longitudinal axis mx. The longitudinal axis mx of the metering element 33 is parallel to the main longitudinal axis x of the device 1. In particular, the longitudinal axis mx coincides with the main longitudinal axis x of the device 1. The metering element 33 is axially and rotationally movable with respect to the storage chamber 15. When the cap 7 is demounted from the device 1, i.e. during an operation of the device 1, the metering element 33 is moved in a distal direction 18. The distal direction 18 is a direction towards a dispensing end of the device. When the cap 7 is remounted onto the device 1, i.e. after an operation was completed, the metering element 33 is moved in a proximal direction 19. The proximal direction 19 is a direction away from the dispensing end of the device. The metering element 33 is rotationally connected to the rotary part 25 by mechanical cooperation with the rotary part 25. Accordingly, the metering element 33 follows rotational movement of the cap 7 and, hence, of the rotary part 25 about the main longitudinal axis x when the cap 7 is mounted onto the device 1 or demounted from the device 1.

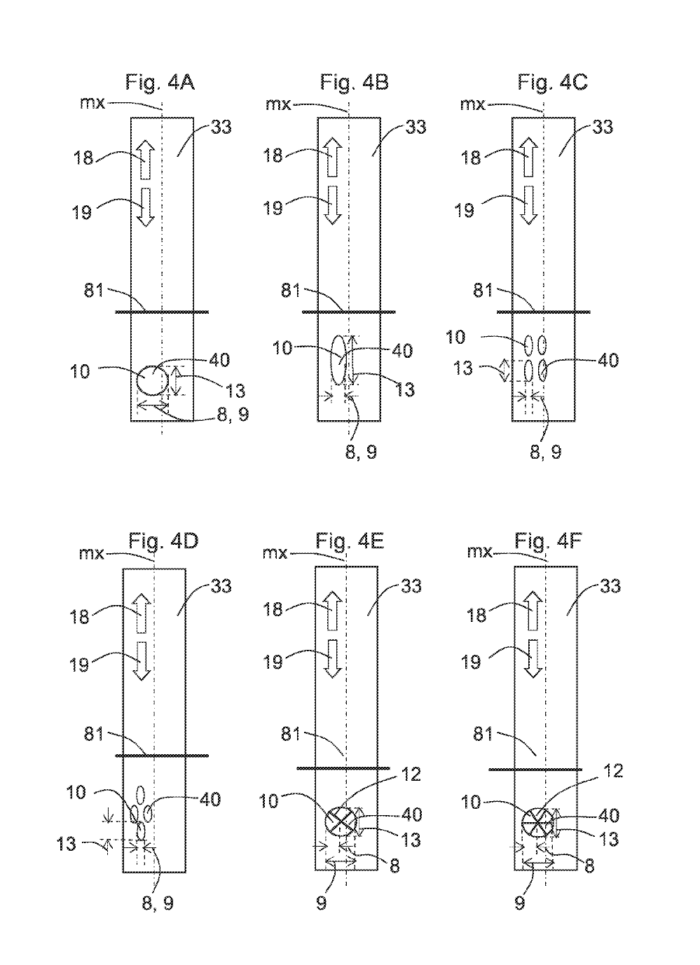

The metering element 33 comprises a metering chamber 40. The metering chamber 40 is located near a proximal end of the metering element 33. The proximal end of the metering element 33 is the end, which his located in the storage chamber 15 when the cap 7 is mounted on the device. The metering element 33 is configured for moving the metering chamber 40 from a first position, wherein the metering chamber is located inside the storage chamber 15, to a second position, wherein the metering 40 is located outside of the storage chamber 15. The metering chamber 40 is configured for measuring and accommodating a sub-quantity 14 of the substance 2 which is to be dispensed during an inhalation action performed by a user. In particular, a sub-quantity 14 of the substance 2 may be transported from the storage chamber 15 to a powder channel 16 via the metering chamber 40. In order to collect a sub-quantity 14 of the substance 2, the metering chamber 40 comprises an opening 10, which will be described in more detail in FIGS. 4A to 4F. As can be seen in FIG. 2, the opening 10 is arranged eccentric with respect to the axis mx on the metering element 33, in order to achieve an adequate filling of the metering chamber 40 with substance 2. In particular, the metering chamber 40 helicoidally moves through the storage chamber 15, thereby gathering a sub-quantity 14 of substance 2. The shape of the opening influences the flow profile of the substance 2 in the powder channel 16. Particularly, the flow profile influences the composition of the fine particle fraction of the substance 2.

For a detailed description of the operation of the metering element 33, it is referred to document WO 2009/065707 A1.

The device 1 further comprises a sealing member 31. The sealing member 31 comprises a main body 32 and a sealing lip 81. The cylindrical portion 25A accommodates the sealing member 31 in its center. In particular, the sealing member 31 is arranged subsequently to the cylindrical portion 25 in a radial inward direction with respect to the housing 3. The sealing member 31 is arranged between the cylindrical portion 25A and the metering element 33. The sealing member 31 is arranged circumferentially around the metering element 33. The sealing member 31 is arranged at that end of the storage chamber 15 which is faced towards the mouthpiece 6. The sealing member is arranged outside of the storage chamber 15.

The sealing member 31 comprises or consists of a rubber material or a similar elastic material. For example, the sealing member 31 comprises a thermoplastic elastomer. The sealing member 31 may be produced by a two-component injection molding process together with the rotary part 25. Alternatively, the sealing member may be produced in a separate manufacturing step. In this embodiment, the sealing member 31 and the rotary part 25 are connected to one another such that the sealing member 31 is secured against axial and rotational movement with respect to the rotary part 25. The sealing member may, for example, be clipped into the rotary part 25.

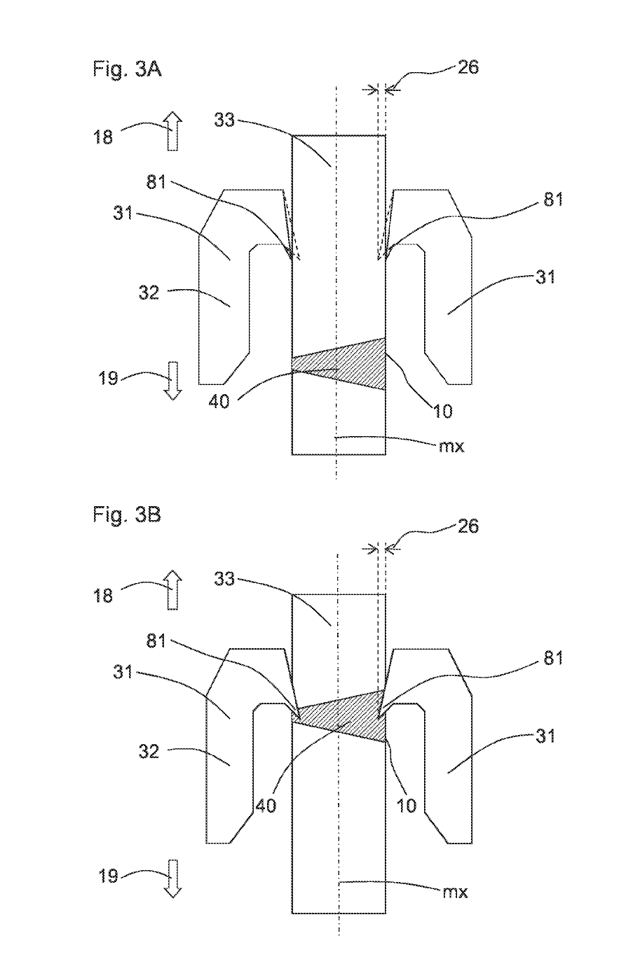

As can be seen in FIG. 3A, when the sealing member 31 mechanically cooperates with the metering element 33, the sealing member 31, respectively the sealing lip 81, is radially deflected. In particular, the sealing lip 81 is deflected in a direction away from the longitudinal axis mx because of the presence of the metering element 33 inside of the sealing member 31. For better exemplification, the undeflected condition of the sealing lip 81 is indicated by dashed lines. The sealing lip 81 is deflected about an excess 26. The excess 26 may have an amount between 0.1 and 1.0 mm. For example, the amount of the excess 26 is 0.5 mm. Due to its elasticity, the sealing lip 81 strives to deflect in a radial direction towards the metering element 33.

FIG. 3B shows a mechanical cooperating of a metering element 33 with a sealing member 31, wherein the metering element 33 comprises an opening 10 having a continuous width 8 being equal to its entire height 13 as a comparison example. When the metering element 33 is axially moved in a first direction 18 such that the metering chamber 40 is moved from the storage chamber 15 to the powder channel 16, the metering chamber 40 has to pass between the sealing lips 81. When the metering chamber 40 is at height of the sealing lip 81, the sealing lip 81 may not be deflected by the presence of the metering element 33 anymore, but may due to its elasticity bend inward in a direction towards the axis mx and immerge into metering chamber 40 through opening 10. Thereby, an amount of the sub-quantity 14 is removed from the metering chamber 40. Thus, an accurate amount of substance 2 does not completely arrive in the powder channel 16. Thereby a wrong dose of medicament is delivered to a user.

A metering element 33 according to FIG. 3B is shown in FIG. 4A. The metering chamber 40 of the metering element 33 has a circular opening 10. The sealing lip 81 is aligned perpendicular to the moving direction of the metering element 33. The shape of the opening 10 allows the sealing lip 81 to immerge into the metering chamber 40.

FIGS. 4B to 4F show different embodiments of a metering element 33. In these embodiments, the opening 10 is shaped such that an immerging of the sealing lip 81 into the metering chamber 40 is impeded. The sealing lip 81 is in each case aligned perpendicular to the moving direction of the metering element 33, while the height 13 of the opening 10 extends perpendicular to the orientation of the sealing lip 81.

FIG. 4B shows a metering element 33 which is similar to the metering element 33 as described in FIG. 4A, apart from the opening 10 being shaped differently. The opening 10 has a continuous width 8 which is less than the entire height 13 of the opening 10. The entire height 13 of the opening 10 extends along the longitudinal axis mx of the metering element 33. The continuous width 8 of the opening 10 extends across the longitudinal axis mx. In particular, the continuous width 8 extends perpendicular to the longitudinal axis mx. In particular, the opening 10 has the form of an ellipse. In FIG. 4B, the continuous width 8 coincides with the entire width 9 of the opening 10. The opening 10 may have a continuous width 8 between 1 mm and 3 mm. For example, the opening 10 has a continuous width of 1.5 mm. Because of the narrow configuration of the opening 10, the sealing lip 81 cannot immerge into the metering chamber 40. Thereby, a removal of an amount of the sub-quantity 14 of a substance 2 from the storage chamber 15 is inhibited.

FIG. 4C shows a different embodiment of a metering element 33. The metering element 33 comprises four openings. In particular, the opening is divided in four segments. The openings are arranged in terms of a rectangle. Each opening 10 comprises a continuous width 8 and an entire height 13. The continuous width 8 of each opening 10 is less than its entire height 13. In FIG. 4C, the continuous width 8 of each opening 10 coincides with its entire width 9. The openings 10 have the form of an ellipse. Each opening 10 may have a continuous width 8 between 0.5 mm and 1.5 mm. For example, each opening 10 may have a continuous width 8 of 1.0 mm. Because of the narrow configuration of each opening 10, the sealing lip 81 cannot immerge into the metering chamber 40. Thereby, a removal of an amount of the sub-quantity 14 of a substance 2 from the storage chamber 15 is inhibited.

FIG. 4D shows an embodiment of a metering element which is similar to the embodiment of FIG. 4C, apart from that the openings 10 are arranged differently. In particular, the openings are arranged in terms of a rhomb.

FIG. 4E shows a further embodiment of a metering element 33. In this embodiment, the metering element 33 comprises a grating 12. The opening 10 is formed circular. By means of the grating 12, the opening 10 is divided such that it comprises four segments. Each segment has a continuous width 8 which is less than the entire height 13 of the opening 10. For example, the grating has the form of an “x”. For example, the grating comprises a filament being placed in the opening 10. In particular, by means of the grating 12, the continuous width 8 of the opening 10 is reduced. The opening 10 comprises a continuous width 8, which is less than its entire width 9. By means of the grating 12, an immerging of the sealing lip 81 into the metering chamber 40 is inhibited.

FIG. 4F shows an embodiment of a metering element which is similar to the embodiment of FIG. 4E, apart from that the opening 10 is divided by a grating 12 such that it comprises six segments. The grating 12 has the form of a star.

In further embodiments, the features of the embodiments of FIGS. 4B to 4F may be combined with each other. For example, an elliptical opening may also comprise a grating.

1-15. (canceled)

16. A metering element for an inhalation device, comprising:
at least one opening being configured to receive a substance, wherein the opening comprises a width and an entire height, wherein the height extends along a longitudinal axis of the metering element and the width extends across the longitudinal axis, and
wherein the continuous width of the opening is less than the entire height of the opening, and
wherein the opening is divided such that it comprises at least two segments.

17. A metering element for an inhalation device according to claim 16, wherein the opening has the form of an ellipse.

18. A metering element for an inhalation device according to claim 16, wherein the metering element comprises at least two openings.

19. A metering element for an inhalation device according to claim 16, comprising at least four openings arranged in terms of a square.

20. A metering element for an inhalation device according to claim 16, comprising a grating being configured to reduce the continuous width of the opening.

21. A metering element for an inhalation device according to claim 20, wherein the grating defines two or more segments of the opening.

22. A metering element for an inhalation device according to claim 16, comprising a metering chamber being configured to receive a sub-quantity of a total quantity of substance via the opening.

23. A metering element for an inhalation device according to claim 22, wherein the opening is shaped such that a removal of substance from the metering chamber is inhibited.

24. An assembly for an inhalation device, comprising a metering element according to claim 16, and a sealing member wherein a removal of substance from the metering chamber upon mechanical cooperation of the metering element with the sealing member is prevented.

25. An assembly for an inhalation device according to claim 24, wherein the sealing member comprises a main body and a sealing lip being aligned perpendicular to a longitudinal axis of the device.

26. An assembly for an inhalation device according to claim 24, wherein the metering chamber is configured such that the sealing member does not immerge into the metering chamber.

27. An assembly for an inhalation device according to claim 24, wherein the metering element is configured to axially move along the longitudinal axis of the device.

28. An assembly for an inhalation device according to claim 24, comprising a storage chamber.

29. An assembly for an inhalation device according to claim 28, wherein the metering element is configured to transport a sub-quantity of substance out of the storage chamber.

30. An assembly for an inhalation device according to claim 28, wherein the metering element is configured to rotate and to move axially with respect to the storage chamber.
